(19) United States
(12) Patent Application Publication
(10) Pub. No.: US 2020/0268442 A1
(43) Pub. Date: Aug. 27, 2020

(54) ENERGY DELIVERY DEVICE FOR ENDOVASCULAR OCCLUSION

(71) Applicant: Cook Medical Technologies LLC, Bloomington, IN (US)

(72) Inventors: Rune T. Paamand, Humlebaek (DK); Per Elgaard, Haslev (DK); Jan M. Kristoffersen, Naestved (DK)

(73) Assignee: Cook Medical Technologies LLC, Bloomington, IN (US)

(21) Appl. No.: 16/721,279

(22) Filed: Dec. 19, 2019

(30) Foreign Application Priority Data
Dec. 20, 2018 (GB) 1820854.6

(51) Int. Cl. A61B 18/14

(52) U.S. Cl. CPC A61B 18/1492; A61B 2018/0041

(57) ABSTRACT

An endoluminal energy delivery device includes a proximal end and a distal end, an electrically conductive core extending from the proximal to the distal end, and an energy delivery tip at the distal end. The energy delivery tip includes a distal core portion and an electrically conductive element disposed coaxially around the distal core portion and electrically coupled thereto in parallel. The distal core portion is made of a first material and the electrically conductive element is made of a second material. The second material has a higher conductivity than the first material. Electrical energy supplied to the delivery device passes simultaneously through the distal core portion and the electrically conductive element.

### CROSS-REFERENCE TO RELATED APPLICATIONS

This application claims priority to Great Britain Application No. 1820854.6, filed Dec. 20, 2018. The contents of Great Britain Application No. 1820854.6 are incorporated by reference in their entirety.

### TECHNICAL FIELD

The present invention relates to an energy delivery device or delivery guide wire for example for occluding or closing a vessel.

### BACKGROUND ART

A number of medical conditions require the closure of blood vessels, including for example, to stop internal organ haemorrhaging, for treating organ or muscle bleeding following trauma, starving organs of oxygen and nutrients in the treatment or containment of cancerous growth, and so on.

Conventionally, vessels have been closed by means of external ligation, which generally must be carried out by an open surgery procedure, carrying with it its associated risks, inconvenience and long patient recovery times. More recent techniques for closing blood vessels involve endoluminally inserting into the vessel or organ one or more occlusion devices, such as microcoils, vascular plugs, gelfoam or liquid embolics so as to obstruct the flow of blood in the vessel. There is a risk of migration of such devices over time, incomplete occlusion and effects of keeping a foreign object in the vessel.

Another recent technique for occluding a vessel is endoluminal ablation. This technique involves the use of an energy delivery terminal which is fed endoluminally into the vessel. Energy is applied to the terminal to cause localised heating to the vessel wall tissue, resulting in vessel contraction and sealing.

Examples of prior art devices can be found in US-2015/0134057, US-2017/0049511, US-2016/20082180, US-2016/0175009, U.S. Pat. Nos. 6,210,408, 5,743,905, 9,364,286, 6,019,757, 9,192,435 and US20150196355.

### SUMMARY OF THE INVENTION

The present invention seeks to provide an improved energy delivery guidewire for occluding a vessel.

According to an aspect of the present invention, there is provided an endoluminal energy delivery device including a proximal end and a distal end; an electrically conductive core extending from the proximal to the distal end; an energy delivery tip at the distal end; the energy delivery tip including a distal core portion and an electrically conductive element disposed coaxially around the distal core portion and electrically coupled thereto in parallel; wherein the distal core portion is made of a first material and the electrically conductive element is made of a second material, wherein the second material has a higher conductivity than the first material; whereby electrical energy supplied to the delivery device passes simultaneously through the distal core portion and the electrically conductive element.

Devices used to perform endoluminal ablation often use conventional guidewires for tracking the energy delivery terminal to the treatment site. These devices typically comprise a good conductor that can connect the energy generator to the energy delivery terminal. This is suitable for large devices where the conductor may be embedded into the device, for example where the core may be used as a conductor. However, such guidewires are often too large for performing selective artery embolization.

Narrow conductors often used in superselective embolization of very small vessels often present increased resistivity and this, together with the desire to maintain mechanical support potentially creates a problem for superselective embolization of very small vessels. Conventionally, small devices used for superselective navigation require cores with good mechanical properties in order to support the device. However, these result in poor conductors which limit the capacity to carry current without self-heating and consequently limit the power that can safely be delivered with the device.

Devices used for superselective navigation often have a tapered mandril design. However, this design increases electrical resistivity of the device, limiting the power that can safely be delivered with the device.

Embodiments of the present invention address the above problems by the device including an electrically conductive core and an electrically conductive element disposed coaxially around the distal core portion and electrically coupled thereto in parallel. The electrically conductive element is made of a material with higher conductivity than the material of the distal core portion, and electrical energy supplied to the device can pass simultaneously through the distal core portion and the electrically conductive element. Thus, the electrically conductive element alleviates the core from current, lowering the heat loss in the core and increasing the total current capacity and maximum power. At the same time, the core, in particular the distal core portion, can provide the device with mechanical support for advancing and supporting the device in narrow vessels.

Preferably, the electrically conductive element is a helical wire coil.

Preferably, the distal core portion is made of a first material and the electrically conductive element is made of a second material, wherein the second material has a greater flexibility than the first material.

Preferably, the distal core portion has a greater stiffness than a stiffness of the electrically conductive element.

Preferably, the distal core portion is made of nickel titanium alloy or stainless steel.

Preferably, the distal core portion is substantially free of copper, silver and gold.

Preferably, the electrically conductive element is made of platinum, gold, silver or alloys thereof.

Preferably, the electrically conductive element is made of or includes a radiopaque constituent.

Preferably, the electrically conductive element is made of a material including tungsten.

Preferably, the endoluminal energy delivery device includes an electrically insulating sheath disposed coaxially around the core from the proximal end of the device to a proximal end of the distal core portion.

Preferably, the core includes a tapered portion tapering towards a distal extremity of the device.

Preferably, the tapered portion is proximal of the distal core portion.

The tapered portion advantageously assists in providing an energy delivery tip which has an energy delivery tip core with a smaller diameter than a diameter of the core at the guidewire proximal portion so as to provide a tip which is soft and floppy for advancing through narrow and tortuous vessels, while the material of the core can ensure that it is not fragile so that it can continue to provide support to the device.

Preferably, the endoluminal energy delivery device includes a second electrically conductive element, the second electrically conductive element being disposed coaxially around the tapered portion.

This advantageously helps in alleviating the tapering core from current, lowering the heat loss in the tapering core and increasing the total current capacity and maximum power.

Preferably, the second electrically conductive element is a wire coil, Preferably, the second electrically conductive element is a tubular coil made of stainless steel.

Preferably, the second electrically conductive element is electrically coupled to the core in parallel; whereby electrical energy supplied to the delivery device passes simultaneously through the tapered portion and the second electrically conductive element.

Preferably, the endoluminal energy delivery device includes an electrically insulating sleeve disposed coaxially over the second electrically conductive element.

Preferably, the endoluminal energy delivery device includes a rounded tip at the distal end of the device, the distal core portion and electrically conductive element being coupled to the rounded tip.

The rounded tip helps to provide an atraumatic end to the device for advancing through a vessel.

Preferably, the rounded tip is electrically conductive, the distal core portion and electrically conductive element being connected together electrically through the rounded tip.

Preferably, the electrically conductive element is coated with an heat conductive material.

Preferably, the energy delivery tip is non-stick with respect to charred blood.

This enables the energy delivery tip to be retracted from the vessel without the risk of tearing and rupturing the vessel wall.

Preferably, the electrically conductive element is coated with a layer of parylene.

Preferably, the electrically conductive element is coated with a layer of chromium nitride (CrN).

Preferably, the device is a radiofrequency ablation electrode.

Preferably, the device is a radiofrequency ablation electrode of a monopolar vessel ablation system.

According to a further aspect of the present invention, there is provided a method of ablating a vessel, including the steps of:

inserting an energy delivery guidewire into a vessel having a vessel wall,

contacting the energy delivery guidewire with the vessel wall at at least two contact points,

delivering energy to the energy delivery guidewire to cause vessel ablation.

Features of the device are applicable to the above method.

According to an aspect of the present invention, there is provided an endoluminal energy delivery device as described above, the endoluminal energy delivery device comprising an energy delivery guide wire, the guide wire including a guidewire proximal portion, a guide wire distal portion and the energy delivery tip, the energy delivery tip extending from the distal portion; the energy delivery tip having a tip length, a proximal tip end and a distal tip end; at least the guide wire distal portion extending in a first direction in an unbiased condition; the energy delivery tip extending in a second direction different from the first direction; wherein the distal tip end is offset from the proximal tip end relative to the first direction by between 10 to 50% of the tip length.

In embodiments of the invention, the distal tip end is offset from the proximal tip end relative to the first direction by between 20 to 50% of the tip length.

In embodiments of the invention, the distal tip end is offset from the proximal tip end relative to the first direction by substantially 20% of the tip length.

In embodiments of the invention, the guide wire distal portion has a straight unbiased configuration.

In embodiments of the invention, the guide wire distal portion has a curved unbiased configuration and the first direction is determined on the basis of a tangent to the curvature of the distal portion.

In embodiments of the invention, the energy delivery tip has a straight unbiased configuration.

In embodiments of the invention, the energy delivery tip has a curved unbiased configuration and the second direction is determined on the basis of a tangent to the curvature of the energy delivery tip.

In embodiments of the invention, the curvature of the energy delivery tip is smooth.

In embodiments of the invention, the curvature of the energy delivery tip is of substantially constant radius.

In embodiments of the invention, the energy delivery tip has a length of between 5 and 20 millimetres.

In embodiments of the invention, the energy delivery tip has a length of between 8 and 12 millimetres.

In embodiments of the invention, the energy delivery tip has a length of substantially 10 millimetres.

In embodiments of the invention, the energy delivery tip is an electrically conductive element and heatable upon the application of electrical energy thereto.

In embodiments of the invention, the proximal and distal portions of the guide wire are provided with an electrically insulating sheath.

In embodiments of the invention, the energy delivery tip includes an energy delivery tip core formed by a distal tip of an electrically conductive core of the guide wire, the core forming the distal and proximal portions of the guide wire.

In embodiments of the invention, at least a portion of the core is tapered.

In embodiments of the invention, the core at the energy delivery tip has a substantially uniform diameter.

In embodiments of the invention, the energy delivery tip core is covered by a wire coil.

In embodiments of the invention, the wire coil is electrically and/or heat conductive.

In embodiments of the invention, the core at the energy delivery tip is formed as a single elongate element.

In embodiments of the invention, a single energy delivery tip extends from the guidewire distal portion.

In embodiments of the invention, the energy delivery tip diameter is no more than about 0.6 mm, optionally no more than 0.54 mm.

In embodiments of the invention, the diameter of the guide wire is no more than about 0.6 mm, optionally no more than 0.546 mm.

In embodiments of the invention, the energy delivery tip is curved in a single plane.

In embodiments of the invention, the energy delivery tip is made of shapable material.

In embodiments of the invention, the energy delivery tip is configured to contact a vessel wall at at least two contact points.

In embodiments of the invention, the energy delivery tip is configured to contact a vessel wall at a first contact point being at the proximal tip end of the energy delivery tip and at a second contact point being at the distal tip end of the energy delivery tip.

In embodiments of the invention, the energy delivery tip is cylinder shaped without any protruding elements,

The features of the claims can be interchanged and combined.

### BRIEF DESCRIPTION OF THE DRAWINGS

Embodiments of the present invention are described below, by way of example only, with reference to the accompanying drawings, in which:

FIG. 1 is a view in side elevation of an embodiment of an endoluminal energy delivery device;

FIG. 2 is a view in side elevation of the device of FIG. 1;

FIG. 3 is a view in side elevation of the device of FIGS. 1 and 2 inserted in a vessel;

FIG. 4 is a cross-sectional view in side elevation of the energy delivery tip of the device of FIGS. 1 to 3;

FIG. 5 is a cross-sectional view in side elevation of the device of FIGS. 1 to 4;

FIG. 6 is a cross-sectional detailed view of the energy delivery tip of the device of FIGS. 1 to 5;

FIG. 7 shows two tables providing data for an embodiment of delivery guidewire using FEP;

FIG. 8 is a cross-sectional view in side elevation of an embodiment of the invention;

FIG. 9 is a finite element analysis diagram showing the heat generated from the energy delivery tip of a device according to an embodiment of the invention;

FIG. 10 is a view in side elevation of the device of FIGS. 1 to 6;

FIG. 11 is a cross-sectional view in side elevation of an embodiment of the invention with dimensions shown;

FIG. 12 is a cross-sectional detailed view of the device of FIG. 11 with dimensions shown;

FIG. 13 is a view in side elevation of the device of FIGS. 11 and 12 with dimensions shown;

FIG. 14 is a view in side elevation of an embodiment of the invention with dimensions shown;

FIG. 15 is a diagram of a monopolar vessel ablation system according to an embodiment of the invention;

FIG. 16 is a front perspective view of an embodiment of delivery guidewire handle; and

FIG. 17 is a cross-sectional view through the handle of FIG. 16.

### DESCRIPTION OF THE PREFERRED EMBODIMENTS

Described below are preferred embodiments of an implantable medical device constructed according to the teachings herein. It is to be understood that the drawings are not to scale and are intended to be merely illustrative of the features and elements of the device and its components. Furthermore, dimensions given below are exemplary only and may be different in different embodiments, for example in accordance with the clinical procedure to be undertaken.

The embodiments described below are of an energy delivery device for endovascular ablation, embolization and/or occlusion. Ablation can include heating a vessel in order to cause coagulation of the blood, embolization and/or vessel contraction, for example to form an occluding plug. The preferred embodiments are designed to be inserted percutaneously into blood vessels. Current is passed to an energy delivery tip to ablate the vessel, in some instances causing contraction and occlusion of the vessel. Some embodiments of the invention can be used for example for embolization of arteries in relation to gastric bleedings, solid organ and muscle trauma and tumors (for example prior to radio/bead embolization).

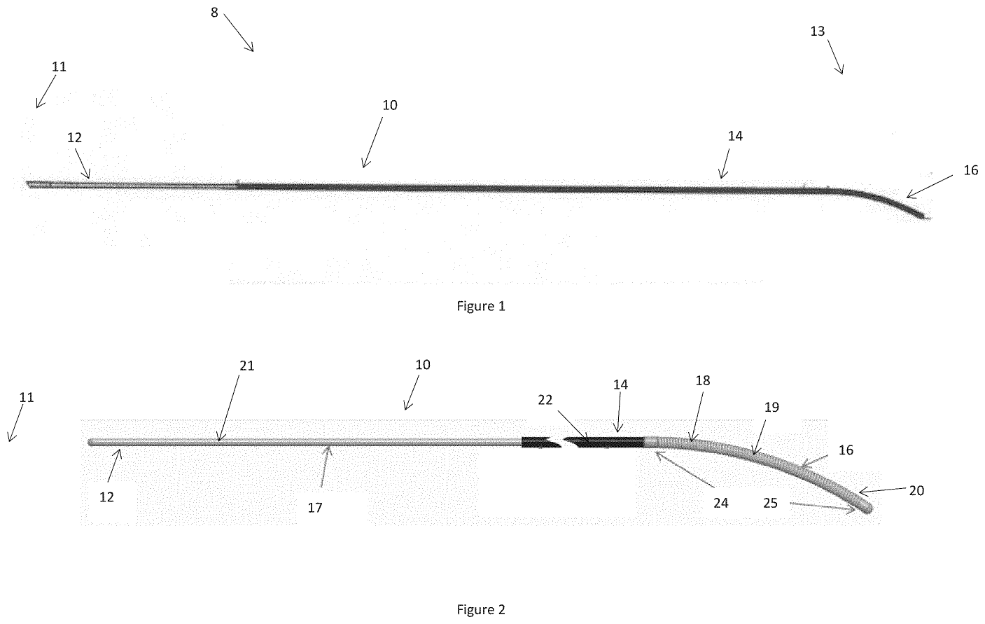

Referring to FIGS. 1 to 3, this shows an endoluminal energy delivery device 8 extending from a proximal end 11 to a distal end 13 and including a guidewire 10. In this embodiment, the device provides a radiofrequency ablation electrode for a monopolar vessel ablation system.

As described above, in order to achieve successful closure of a vessel, it is desirable that there be contact between the vessel tissue and the energy delivery terminal. Producing delivery devices that can achieve this is difficult and previous devices which use self-expanding mechanisms or inflatable balloons may be unsuitable especially for smaller and more delicate vessels. Embodiments of the present invention address this by providing a fully capable energy delivery guidewire 10 with a defined tip geometry that ensures wall contact in at least two locations.

The guidewire 10 includes a guidewire proximal portion 12, a guidewire distal portion 14 and an energy delivery tip 16 extending from the distal portion 14. The tip 16 has a tip length, a proximal tip end 18 and a distal tip end 20. The guidewire distal portion 14 extends in a first direction in an unbiased condition and the energy delivery tip 16 extends in a second direction different from the first direction, the distal tip end 20 being offset from the proximal tip end 18 relative to the first direction by between 10 to 50% of the tip length, preferably at least 20% of the tip length, and in this embodiment by substantially 20% of the tip length. This can enable the delivery tip 16 to engage the vessel wall at two points. Advantageously, the energy delivery tip 16 is configured to engage a vessel wall at or near the proximal 18 and distal 20 tip ends as these are regions of increased heat generation, as explained below.

The geometry also ensures that the device may function as a guidewire while effectively delivering energy to the target vessel wall, enabling the device to achieve superselective embolization of very small vessels. The device does not require a microcatheter to track to the treatment target, making it suitable for smaller vessels.

In the embodiment of FIGS. 1 to 3, the energy delivery tip 16 is electrically conductive and extends from the guidewire distal portion 14. The guidewire 10 proximal of and including the guidewire distal portion 14 extends in the first direction, substantially longitudinally with the longitudinal axis of the device. The energy delivery tip 16 is curved and extends in a second direction different from the first direction, the second direction being defined by a tangent, in this case also by a chord, of the curve of the energy delivery tip 16 as described below. The energy delivery tip 16 assumes this shape when in an unbiased condition, in other words it assumes the curved shape when in a relaxed state.

In this embodiment, the energy delivery tip 16 is flexible, allowing it to more easily navigate through the tortuous routes of the vessels. However, the energy delivery tip is resilient such that it tends to adopt the curved shape. In particular, the delivery tip maintains its curved shape in a vessel. The guidewire proximal of the energy delivery tip, on the other hand, tends to a straight configuration.

In this embodiment, the energy delivery tip 16 is curved with a constant radius of curvature although in other embodiments the radius of curvature can vary. Preferably, the energy delivery tip is curved throughout a major part of its exposed length, in this embodiment it is curved throughout its whole length. In this embodiment, the energy delivery tip curves in a single plane. In other words, the tangent lines defined by the curvature of the energy delivery tip 16 lie in a single plane. In this embodiment, the energy delivery tip 16 is curved such that the longitudinal components of the tangent lines are in the same sense; in other words, the energy delivery tip 16 does not curve back or bend back on itself and each part of the length of the energy delivery tip contributes to the offset. In this embodiment, the curvature of the energy delivery tip is smooth.

The energy delivery tip 16 has a minimum tip stiffness so that it is not completely straightened by the blood flow when the device is in a vessel. The minimum tip stiffness is such that the energy delivery tip maintains contact with the vessel at two locations, preferably at the or near the distal tip end 20 and the proximal tip end 18. The minimum tip stiffness may be defined as a minimum cross section area of the core, or by a minimum spring force (deflection force) as discussed below.

In this embodiment, the device is configured to be used in superselective embolization of very small vessels, vessels typically with a reference vessel diameter of no more than 2.5 mm, in particular peripheral and visceral arteries. The device is designed to be inserted percutaneously into an arterial blood vessel to stop blood flow through the target vessel by means of delivering energy to the vessel wall and creating a thrombus and fibrotic response.

In this embodiment, the energy delivery tip 16 includes two solder points 24, 25, solder point 24 being on the proximal tip end 18 and solder point 25 being on the distal tip end 20 of the energy delivery tip 16.

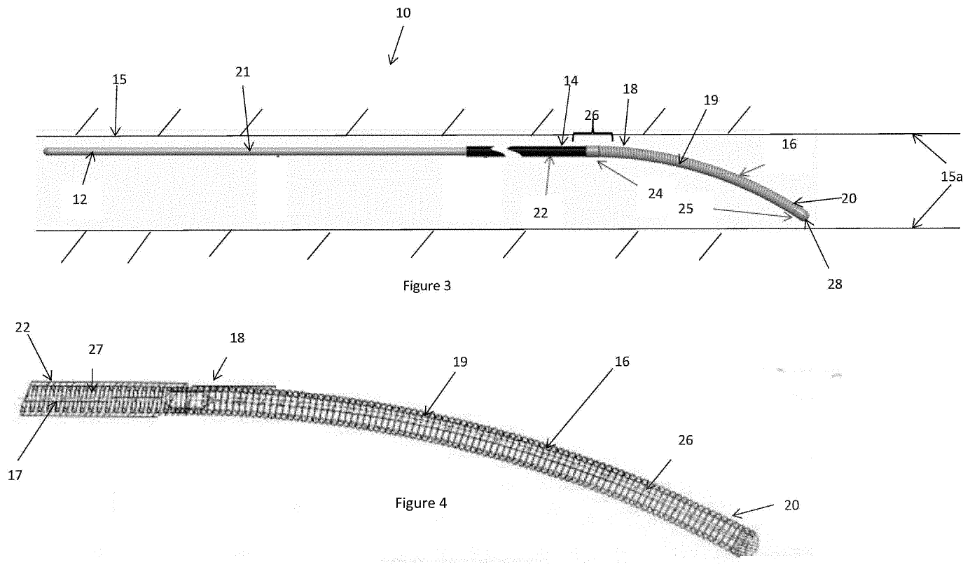

FIG. 3 shows the delivery guidewire 10 inserted into a blood vessel 15. As shown by this Figure, the curved shape of the energy delivery tip 16 ensures that it contacts or engages the vessel wall 15a at two contact points, in this instance one contact point 26 being at the most proximal region of the energy delivery tip 16 and the other contact point 28 being at the distal tip end 20. In practice, the two contact points 26, 28 include the solder points 24 and 25 which provide smooth electrically conductive contact points for the vessel wall 15a.

In the embodiments of FIGS. 1 to 3, a single energy delivery tip 16 extends from the guidewire distal portion 14. This enables the device to be particularly suited to small vessels.

As has been described above, the increased resistivity of narrow conductors and the desire to maintain mechanical support potentially creates a problem for superselective embolization of very small vessels. Conventionally, smaller devices used for superselective navigation require cores with good mechanical properties in order to support the device. However, these result in poor conductors which limit the capacity to carry current without self-heating and consequently limiting the power that can safely be delivered with the device. Embodiments of the present invention address this by the device including an electrically conductive guidewire core 17, which has good mechanical properties for advancing and supporting the device to the target vessel, and, at a distal core portion 26, an electrically conductive element 19 disposed coaxially around the distal core portion 26 and electrically coupled thereto in parallel. The electrically conductive element 19 is made of a material with higher conductivity than the material of the distal core portion 26, and electrical energy supplied to the device can pass simultaneously through the distal core portion 26 and the electrically conductive element 19. Thus, the electrically conductive element 19 alleviates the core from current, lowering the heat loss in the core and increasing the total current capacity and maximum power. At the same time; the core, in particular the distal core portion 26, can provide the device 8 with mechanical support for advancing and supporting the device in narrow vessels.

FIG. 2 is a detailed view of the delivery guidewire 10. The guidewire 10 includes the core 17 which extends from the proximal 11 to the distal 13 ends of the device 8. The guidewire core 17 includes the distal core portion 26 (not shown in FIGS. 1-3 but shown in FIGS. 4-6). Preferably, the guidewire core 17 including the distal core portion 26 is a unitary or monolithic structure. Preferably there is a single core and a single core material. In this embodiment, the core 17 is made of Nitinol and includes a proximal core portion 21 being a mandril with a diameter of 0.015″. However, in other embodiments the core 17 can be made of any material which provides good mechanical properties, such as stainless steel or nickel titanium alloys; in order to be able to advance and support the device even when provided with only a very narrow cross-section.

The guidewire core 17 can have different dimensions from those above, as appropriate for the medical procedure. However, preferably; the diameter of the proximal core portion 21 is as thick as possible, while still compliant with the overall device outer diameter (described in further detail below) in combination with insulation, if such is used, in order to provide good shaft support.

The core preferably does not include typical good conductor materials such as copper, silver, or gold, in view of their poor mechanical properties.

The electrically conductive element 19 can be made of any good conductor, such as platinum, gold, silver, or alloys of these. In addition, the electrically conductive element 19 can be made of or include a radiopaque material. Preferably, the radiopaque material makes the device fluoroscopically visible under non-magnified imaging. In this embodiment, the electrically conductive element 19 is made of platinum with 2-10% tungsten.

In this embodiment, the electrically conductive element 19 is in the form of a helical wire distal coil which coils around the distal core portion 26 and which extends from the proximal tip end 18 to the distal tip end 20. However, other arrangements can be used in other embodiments.

FIG. 4 shows a cross sectional view of the energy delivery tip 16. The distal coil 19 can be seen coiled around the distal core portion 26, which in this embodiment is an energy delivery tip core 26, and extends from the proximal tip end 18 to the distal tip end 20. In this embodiment, the distal coil 19 and the delivery tip core 26 are included in the energy delivery tip 16. Adjacent coil windings of the distal coil 19 are tightly wound with space between the coil windings of about 5%±2.5% of the wire diameter to give the distal coil 19 some flexibility, although different spacing or no space between windings can be provided in other embodiments.

Embodiments of the present invention also include a proximal electrically conductive element 27 disposed coaxially around the guidewire core 17 so that electrical energy supplied to the device 8 can pass simultaneously through the guidewire core 17 and the proximal electrically conductive element 27.

An advantage of the proximal electrically conductive element 27 is that it can be electrically coupled in parallel with the guidewire core 17 in a similar manner to the distal electrically conductive element 19 and the distal core portion 26. Accordingly, the proximal electrically conductive element 27 can alleviate the guidewire core 17 from current, lowering the heat loss in the guidewire core 17 and increasing the total current capacity and maximum power. In this embodiment, the proximal electrically conductive element 27 is in the form of a tubular proximal coil of electrically conductive wire.

In this embodiment, adjacent coil windings of the proximal coil 27 are tightly wound with space between the coil windings of about 5%±2.5% of the wire diameter to give the proximal coil 27 some flexibility, although different spacing or no space between windings can be provided in other embodiments.

In this embodiment, the proximal 27 and distal 19 coils have substantially the same outer diameter. However the skilled person will appreciate that, in other embodiments, the proximal 27 and distal 19 coils may have different outer diameters.

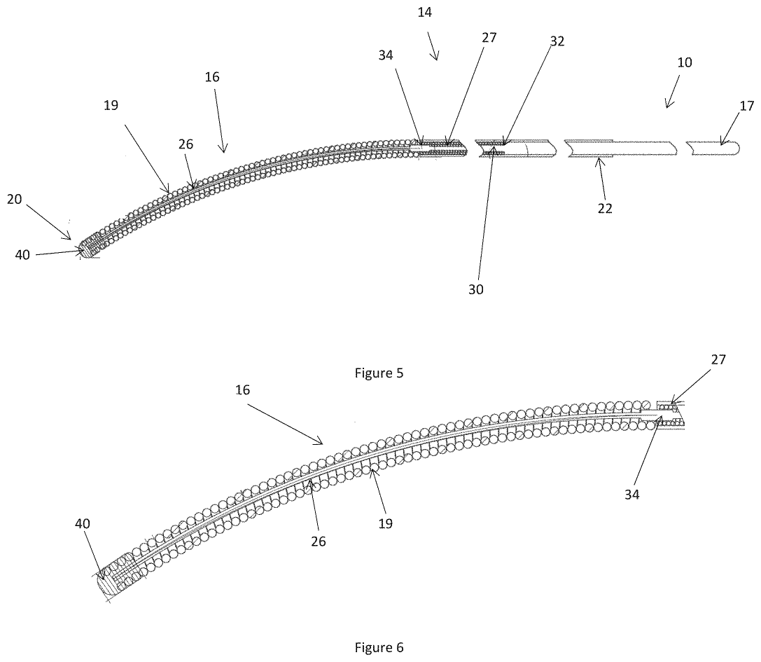

FIG. 5 shows a cross sectional detailed view of the delivery guidewire 10 and the energy delivery tip 16. In this embodiment, the guidewire core 17 including the energy delivery tip core 26 is formed as a single elongate element, with the energy delivery tip core 26 formed by the distal tip of the guidewire core 17, the core 17 forming the distal and proximal portions of the guidewire 10.

In this embodiment, the guidewire core 17 includes a tapered portion 30 at the guidewire distal end 14, although this is not necessary in every embodiment. A stop shoulder 32 is also provided proximal of the tapered portion 30. The stop shoulder 32 provides a step-change in diameter of the core 17, providing a shoulder to limit proximal movement of the proximal coil 27 as described below. In this embodiment, the guidewire core 17 also includes an enlarged centering element 34 at the proximal end of the distal core portion 26.

The tapered portion 30 is located between the stop shoulder 32 and the centering element 34. In this embodiment, the core 17 tapers from the stop shoulder 32 to the centering element 34; however, in other embodiments the tapered portion may extend for only some of the core between the stop shoulder 32 and the centering element 34, The tapered portion 30 may be frustoconical in shape. In this embodiment, the tapered portion 30 has a length of 69.7 mm±0.2 mm. It may be about 70 mm. The tapered portion 30 may provide an area of varying stiffness of the core 17.

The tapered portion assists in providing an energy delivery tip 16 which has an energy delivery tip core 26 with a smaller diameter than a diameter of the core 17 at the guidewire proximal portion so as to provide a tip which is soft and floppy for advancing through narrow and tortuous vessels, while the material of the core can ensure that it is not fragile so that it can continue to provide support to the device.

In this embodiment, the tapered portion 30 tapers from a diameter at its proximal end of about 0.23 mm (possibly down to 0.22 mm) to a diameter at its distal end of 0.06 mm±0.01 mm.

The centering element 34 has a body portion with a diameter which is greater than a diameter of a distal end of the tapered portion 30, but less than a diameter of a proximal end of the guidewire core 17 or of the stop shoulder 32, which in this embodiment is 0,381 mm±0.005 mm. In this embodiment, the outer diameter of the centering element 34 is 0.23 mm, possibly down to 0.22 mm. The centering element 34 in this embodiment has a length of 0.6 mm±0.05 mm.

The energy delivery tip core 26 has a uniform outer diameter, which is typically slightly less than, but may be the same as, the diameter of a distal end of the tapered portion 30. In this embodiment, the diameter of the delivery tip core 26 is 0.05 mm±0.005 mm. The distal core portion does not taper in this embodiment, but rather has a substantially uniform diameter. However, it may taper in some embodiments.

The energy delivery tip 16 has in this embodiment a length of between 5 and 20 millimetres, preferably of between 8 and 12 millimetres, and preferably still of 10 millimetres. The energy delivery tip is the extent of the tip of the guide wire that forms an active electrode and provides energy delivery, and in this embodiment is from about half way along the centering element 34 to the distal tip end 20, in other words from the proximal end of the distal coil 19 to the distal tip end 20.

In this embodiment, the delivery tip core 26 has a length of about 9.7 mm, possibly up to 10.2 mm,

The electrically conductive element 27 can be electrically coupled in parallel with the tapered portion 30 so that electrical energy supplied to the device can pass simultaneously through the tapered portion 30 and the proximal electrically conductive element 27. Accordingly, the proximal electrically conductive element 27 can alleviate the tapering core from current, lowering the heat loss in the tapering core and increasing the total current capacity and maximum power.

In this embodiment, the proximal coil 27 is made of stainless steel wire. However, other conductive materials may be used, such as platinum alloy. The proximal electrically conductive element 27 may be made of a material with higher conductivity than the material of the core 17, in particular of the tapered portion.

In this embodiment, at least part of the proximal coil 27 is disposed coaxially around the tapered portion 30 so that it is disposed around at least the distal part of the tapered portion where the diameter of the tapered portion is narrowest. Part of the proximal coil 27 preferably extends distally of the distal end of the tapered portion and over the centering element 34 and may be attached thereto.

In this embodiment, the length of the proximal coil 27 is 70 mm, possibly down to 69.5 mm. Preferably, the length of the proximal coil 27 is at least as long as the length of the tapered portion 30 so that the proximal wire coil is disposed around the tapered portion 30 along the whole length of the tapered portion 30. In this embodiment, the length of the proximal coil 27 equals the length of the tapered portion 30+half the length of the centering element 34.

The proximal coil 27 can at its proximal end be electrically connected to the guidewire core 17. In addition to being attached to the centering element 34 (as described above), the proximal end of the proximal coil 27 is, in this embodiment, also connected by being soldered to the guidewire core 17 adjacent to the shoulder 32; however, in other embodiments connection may be by other means such as laser welding. In this embodiment, the soldering extends for 0.5 mm±0.2 mm. The connection covers at least three windings of the proximal coil 27.

The proximal coil 27 is, at its distal end; electrically and physically connected to the electrically conductive element, or distal coil 19. In this embodiment, the coils are connected by being soldered together at solder point 24; however, in other embodiments they may be connected by other means such as laser welding. The soldering 24, which may in other embodiments be a different form of connection, is provided covering at least three coil windings on each of the proximal and distal wire coils 27, 19. In this embodiment, the soldering 24 extends for 0.7 mm, possibly up to 0.9 mm.

Typically, the soldering 24 is positioned over the centering element 34 and in some embodiments is soldered or otherwise attached thereto. Accordingly, the proximal and distal wire coils 19, 24 both extend partially over the centering element 34, may be attached thereto, and have inner diameters at least as great as an outer diameter of the centering element 34 to allow this. The solder point 24 provides a smooth covering over windings of the distal coil 19.

In this embodiment; the distal coil 19 has a larger wire diameter than the proximal coil 27. The distal coil 19 is made from a wire with a diameter of about 0.13 mm, for example 0.13 mm-0.15 mm or 0.1224-0.1325; while the proximal coil 27 has a wire diameter of about 0.07 mm, for example 0.065 mm 0.0725 mm. These dimensions give advantageous mechanical properties when combined with a nitinol guidewire core 17.

Preferably, the distal coil 19 is longer than the energy delivery tip core 26 so that it is disposed along the full length of the energy delivery tip core 26 and can extend over and possibly be attached to the centering element 34 as discussed above. In this embodiment, the length of the distal core portion+half the length of the centering element 34 equals the length of the distal coil 19, which in this embodiment is 10 mm±0.5 mm,

In some embodiments, the energy delivery tip is cylinder shaped and is without any protruding elements. This prevents the energy delivery tip from getting trapped in the occlusion following ablation and enables the energy delivery tip to be retracted from the vessel without the risk of tearing and rupturing the vessel wall.

The electrically conductive element or distal coil 19 is coated with a heat conductive material. This is not necessary in every embodiment, but can provide advantages, for example for non-stick properties. In preferred embodiments, the surface of the tip 40 is non-stick with respect to charred blood. In some embodiments, the heat conductive material can be stainless steel, which has shown non-stick properties in heated blood. In other embodiments, the heat conductive material can be a chromium nitride (CrN) layer (for example from Teknologisk Institut) which has been shown to be better than stainless steel and is particularly advantageous where the electrically conductive element 19 is made of platinum. In other embodiments, the heat conductive material can be a layer of parylene, which can be used as a tip coating in very thin layers. Parylene can also provide poor adhesion or non-stick properties to the tip 40 with respect to coagulant, blood and charred blood. This aids in preventing the energy delivery tip from sticking to the vessel wall and enables the energy delivery tip to be retraced from the vessel without risk of tearing and rupturing the vessel wall.

In this embodiment, the device 8 includes a rounded tip 40 at the distal end of the device. The rounded tip 40 is electrically conductive, and the energy delivery tip core 26 and the electrically conductive element 19 are coupled to the rounded tip 40 to be electrically coupled together through the rounded tip 40. The rounded tip 40 helps to provide an atraumatic end to the device for advancing through a vessel.

The rounded tip 40 can be provided in a variety of ways, such as with a cap, welding, or solder. In this embodiment, the rounded tip 40 is provided by soldering together the distal ends of the energy delivery tip core 26 and the electrically conductive element 19 such that the solder surrounds the outer diameter of the device and forms a rounded tip of solder material at the distal end. In this embodiment, the solder of the rounded tip 40 covers at least three windings of the coil 19. In this embodiment, the rounded tip 40 has a length of between about 0.5 mm and 0.8 mm, preferably about 0.8 mm. In this embodiment, the rounded tip provides a solder point which provides a sooth covering over windings of the distal coil 19.

The device current capability is dependent on the cross-section area and tip surface area. Insufficient cross-section can lead to self-heating while small tip surface can result in premature impedance shift from charring. Preferably, the device is configured to reach impedance shift at mid-level power submerged in blood in more than 10 and less than 60 seconds. To achieve this, preferred embodiments have >0.020″ tip outer diameter and >8 mm tip length. Furthermore, a nitinol mandril with >0.0135″ outer diameter is sufficient for 40 W power.

The smallest diameter of the core 17 is preferably defined as a function of the rated power capacity. For example, for a core material with a specific conductivity of 1.22×10⁶S/m, such as Nitinol, preferred embodiments have the following minimum diameters for the smallest diameter of the core 17, which will typically be in the energy delivery tip core 26.

| Power Capacity | Minimum diameter |
|---|---|
| 25 W | 0.065 mm |
| 35 W | 0.080 mm |
| 50 W | 0.095 mm |

The guidewire distal portion 14 in this embodiment includes insulation 22, which may be in the form of an insulating sleeve, which surrounds the guidewire core 17 and the proximal coil 27. In embodiments including the insulation 22, it extends from the proximal tip end, in this case the distal end of the proximal coil 27 at least to the proximal end of the proximal coil 27, and in some embodiments proximally thereof. In this embodiment, the length of the insulation 22 is 1787 mm±5 mm. In some embodiments, the length of the insulation is defined by the length of the device, minus the length of the energy delivery tip 16, minus the length of the core 17 outside and proximal of the insulation.

The insulation 22 may be a polymer jacket. The insulation 22 may be a PET shrink tubing or any other suitable insulating tubing known to the skilled person, for example polymer such as FEP or PTFE. In this embodiment, the insulation 22 has a wall thickness of about 0.0025″ (about 0.0635 mm) (for example it may range from 0.0255 mm to 0.0765 mm). In one embodiment, the insulation thickness is about 0.064 mm, for example 0.057 mm-0.070 mm. In some embodiments, the insulation is thick enough to comply with standard 60601-2-2.

In some embodiments, the insulation thickness is determined using the following equation derived from 60601-2-2:

$$T = \frac{OD}{2} [ 1 - exp ( - \frac{{U}_{peak} * 4 {π}^{2} * f * {ɛ}_{0} * K}{\sqrt{2} * 2 \text{,} 13 * {10}^{8}} ) ]$$

where, in one embodiment:

Upeak: rated peak voltage, 115V

f: RF frequency, 500 kHz

ε₀: vacuum permittivity, 8,854 [F/m]

K: dielectric constant, 3,3 for PET; 2,1 for FEP

For PET: 0,059 mm (0.00235′)

For FEP: 0.038 mm (0.00151″)

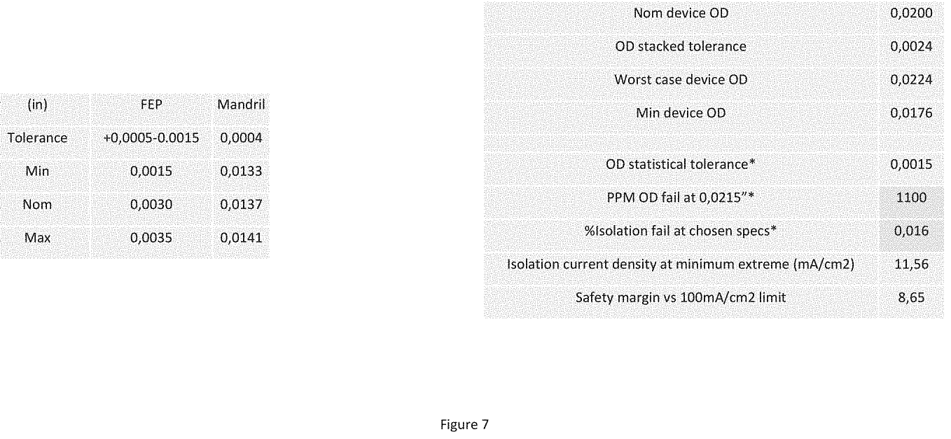

The above calculations result in a margin of safety of 100/11.46. This margin is defined in 60601-2-2 to allow for worst-case variation. This may be redundant double-safety for this well defined use-case. FIG. 7 provides exemplary data for an embodiment using FEP. The design of FIG. 7 preferably has full control of the outer diameter, preferably by use of gauge ring, although full control is preferably already a consequence of manual soldering and not to be avoided. The isolation test preferably passes 60601-2-2 with a representative sample; a large safety margin is added to the test and a failure within spec is considered safe.

In preferred embodiments, the device within the sheath 22 has the lowest possible friction inside the sheath 22 and vessels, for example to allow the guidewire and energy delivery tip 16 to slide within the sheath 22 and within vessels. In addition, the sheath 22 can be low friction to allow the sheath 22 to slide within vessels.

In this embodiment, the overall length of the device is 1800 mm±5 mm, although it may have different dimensions in other embodiments. For example, it may be longer if required for contact with the handle (described further below).

The length of the core 17 proximal of the insulation 22 is preferably defined by the handle contact geometry. In this embodiment, it is 3 mm±1 mm.

FIG. 6 shows a cross-sectional detailed view of the energy delivery tip 16. The proximal coil 27 can be seen extending over a proximal half of the centering element 34. Similarly, the distal coil 19 can be seen extending over a distal half of the centering element 34 and across the energy delivery tip core 26 up to the rounded tip 40.

In this embodiment, the distal coil 19 is made of a material which has a greater flexibility than the material of the guidewire core 17, and the delivery tip core 26 has a greater stiffness than a stiffness of the distal coil 19. This allows the distal coil 19 to follow the curve of the energy delivery tip core 26. The curved shape of the energy delivery tip 16 results in the rounded tip 40 being offset from the centering element 34. In this embodiment, the energy delivery tip 16 is curved with a constant radius of curvature over the full length of the energy delivery tip 16 and the distal tip end, which in this embodiment is the rounded tip 40, is offset from the proximal tip end relative to the first direction by 3 mm+0.5/−0.00 mm when the length of the energy delivery tip is 10 mm±0.5 mm. The flexible distal coil 19 can complement the flexibility caused by the tapering core and help to make the outer diameter of the device at the energy delivery tip more uniform in spite of the tapering core. However, the energy delivery tip core 26 still provides stiffness to advance and support the device 8 in the vessel.

In this embodiment, the stiffness of the energy delivery tip can be approximated as a deflection force equal to that of a nitinol stylet of radius 0.04 mm, tip length 10 mm. The deflection force (P) can be calculated by the following equation.

$$P = \frac{3 \cdot δ \cdot E \cdot I}{{L}^{3}}$$

where:

δ: Deflection (def. to 0.5 mm)

E: Elastic modulus (82 GPa for austenitic Nitinol)

I: Moment of inertia

L: Length (10 mm)

For a rod fixed at one end the moment of inertia is:

$$I = \frac{π}{4} \cdot {r}^{4}$$

where r: Radius (0.04 mm)

Therefore, the resulting equivalent force is:

$$P = \frac{3 \cdot 0.5 mm \cdot 82 GPa \cdot \frac{π}{4} \cdot {( 0.04 mm )}^{4}}{{( 10 mm )}^{3}}$$

P=25 mN (milli Newton)

In this embodiment, the proximal coil 27 has an outer diameter of 038 mm±0.01 mm and the outer diameter of the distal wire coil 40 is 0.53 mm±0.01 mm. Therefore, in such embodiments, the energy delivery tip has a maximum diameter of 0.54 mm. Preferably, the outer diameter of the distal wire coil 40 does not exceed the total device outer diameter, which in this embodiment is 0.546 mm.

In this embodiment, the inner diameter of the proximal coil 27 is 0.24 mm, possibly down to 0.23 mm.

The maximum outer diameter of the core 17 does not, in combination with the insulation 22, exceed a total device outer diameter. Similarly, the outer diameter of the proximal coil 27 does not, in combination with the insulation 22, exceed a total device outer diameter. In some embodiments, the shaft has a nominal diameter of 0.508 mm to never exceed the max, device outer diameter of 0,546 mm if both core outer diameter 0.38 mm±0.005 and insulation thickness 0,064 mm+0,006-0,007 are on max tolerance ˜0,525 mm. In some embodiments, the total device outer diameter is 0.546 mm, the insulation wall thickness is 0.0765 mm, and the maximum outer diameter of the core 17 is 0.385. In such embodiments, insulation thickness*2+maximum core outer diameter=(0.0765 mm*2)+0.385 mm=0.1524 mm+0.385 mm=0.538 mm, which is less than the total device outer diameter. In some embodiments, the total device outer diameter may be 0.546 mm, the insulation maximum outer diameter may be 0.140 mm and the maximum mandril outer diameter may be 0.385; 0.140+0.385=0.525 mm.

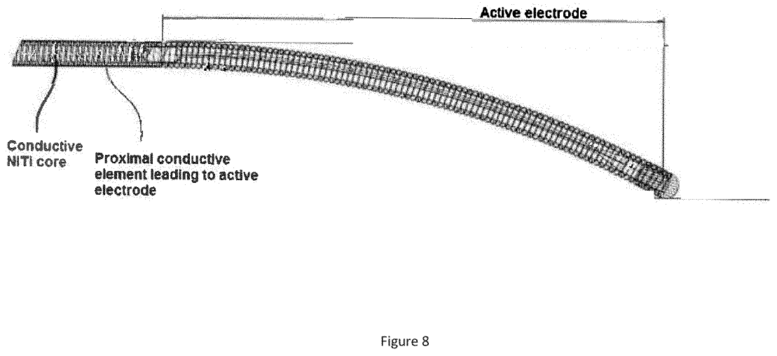

FIG. 8 shows an embodiment of the endoluminal energy delivery device.

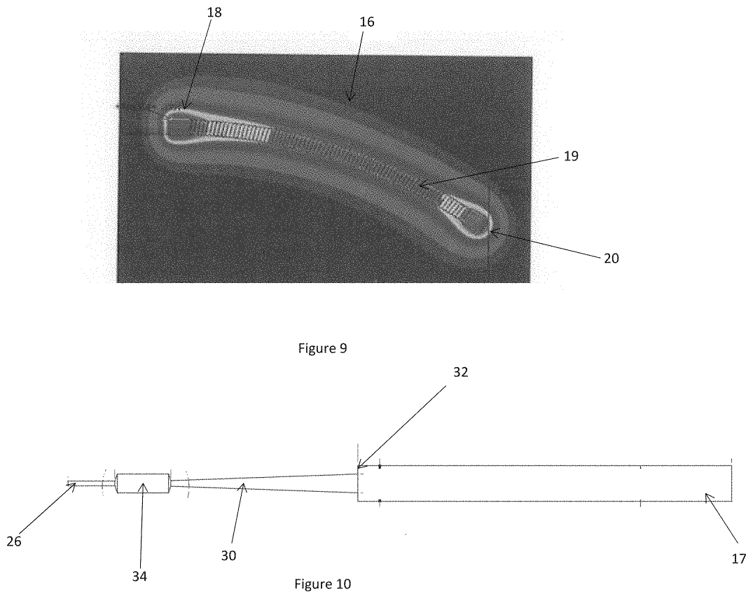

FIG. 9 shows a finite element analysis diagram showing the heat generated from the energy delivery tip 16 when current is passed through it. FIG. 9 illustrates the “dogbone” shape of an electrical field due to the tip 40 effectively acting as an antenna. Two areas of increased energy and hence heat generation can be seen, at the proximal tip end 18 and the distal tip end 20. Although not wishing to be bound by theory, the inventors believe that edge effects result in current distributions and consequently energy delivery at these two points. In the present embodiment, these points are located at about halfway along centering element 34 and at the rounded tip 40, in other words at the proximal tip end and the distal tip end. Because of the advantageous offset of the distal tip end 20 from the proximal tip end 18, the energy delivery tip contacts the vessel wall at contact points 26, 28 at or near the vicinity of the proximal tip end 18 and the distal tip end 20, respectively, thereby maximising heating to the vessel wall and increasing the efficacy of heating the vessel wall, leading to efficient vessel ablation.

In other embodiments, the energy delivery tip 16 may contact the vessel wall at any point along the tip 16, that is, not necessarily at the proximal tip end 18 and the distal tip end 20. Heating to the vessel wall from any point along the tip 16 will still occur, although may be at a reduce efficacy compared to heating from the proximal tip end 18 and the distal tip end 20.

In preferred embodiments, the device produces limited self-heating even during prolonged max-power use, and the internal heat produced is not such as to compromise the device structure.

In embodiments, the device may be configured to comply with ISO11070 and ISO10993.

In preferred embodiments, the device impedance during normal operation (monopolar, submerged in blood) does not exceed 120 Ohms at 500 kHz, and the device is capable of outputting 45 Watts of power for 30 seconds.

FIG. 10 shows a detailed view in side elevation of the guidewire 10 with the proximal 27 and distal 19 coils removed so as to show the guidewire core 17, the tapered portion 30 and the distal core portion 26.

In this embodiment, at least a distal end of the centering element 34 tapers from the body portion to the distal core portion 26. In this embodiment, the taper is 70°±15°. Some embodiments can omit this taper; however, this is not preferred as the taper can assist in making the enlarged portion atraumatic. In some embodiments, a taper can be provided at a proximal end of the enlarged portion to taper from the body portion to the core 17, to assist in making the enlarged portion atraumatic for withdrawal.

Although in the above described embodiments the guidewire is manufactured with the offset, in some embodiments, the device may be manufactured straight but have shapable properties, allowing a physician to create the desired offset (for example by way of a curved shape as discussed above) to more specifically match the target (for example smaller or larger curvature depending on vessel size). Such devices may be made with shapable material. Such devices may have a nitinol or stainless steel core with a construction that allows for the sharp curvatures required for shaping the device as is known to the skilled person.

Irrespective of how first and second directions are provided, the guidewire is configured to tend to and maintain the offset configuration in situ in a vessel.

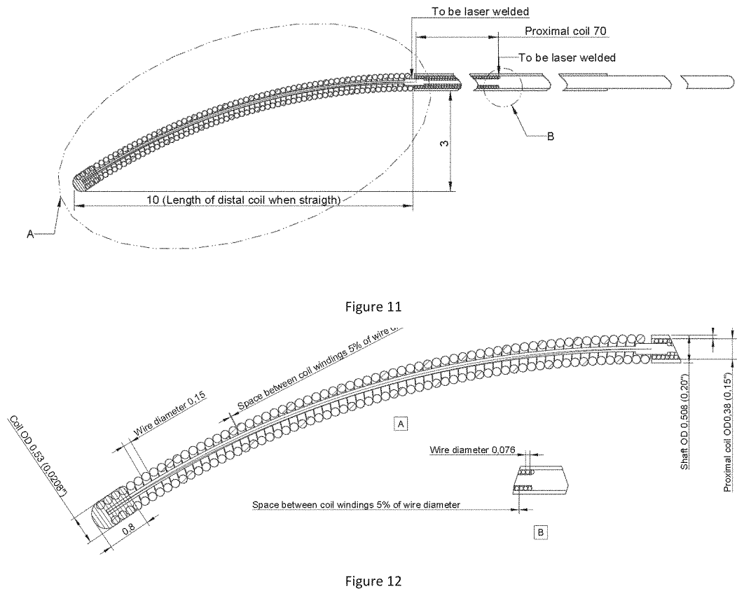

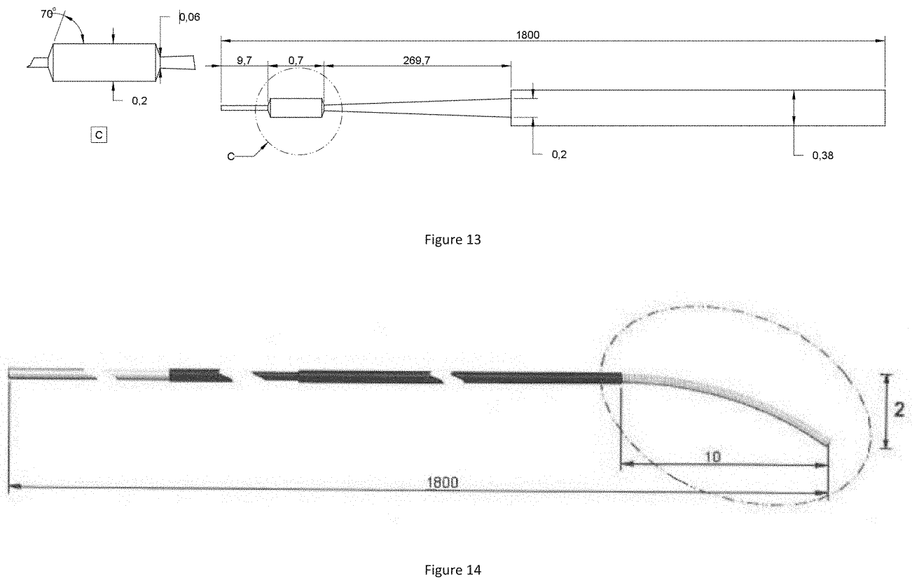

FIGS. 11 to 13 show an embodiment of the endoluminal energy delivery device with some dimensions shown.

FIG. 14 shows an embodiment of the endoluminal energy delivery device with some dimensions shown.

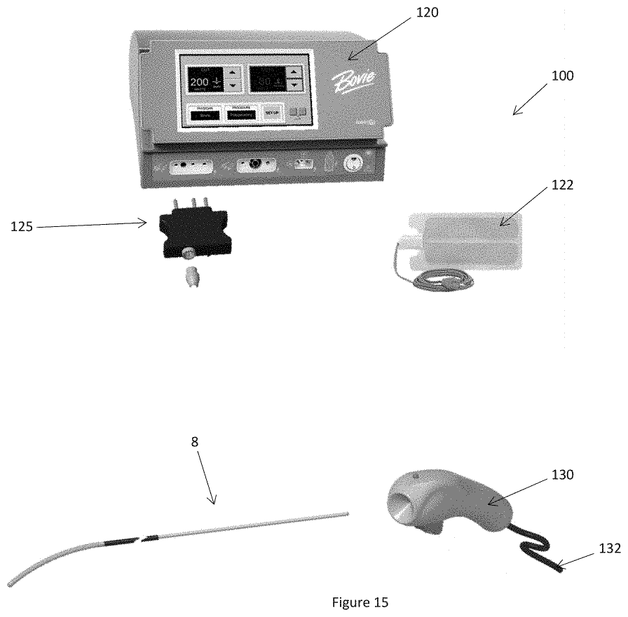

FIG. 15 shows a monopolar vessel ablation system 100 including a device 8 as described above. The system includes a power supply unit 120 providing an RF hub, and this can be a frontend to an electrosurgical unit, for example of a type commercially available from companies such as Bovie, Valleylab or Erbe, or can be a standalone generator. The system 100 also includes a reference pad 122 for being placed in contact with a patient's skin in order to operate with the device 8 to cause ablation, occlusion and/or embolization in a vessel. The reference pad is a dispersive electrode with a large surface area electrically connectable to the power supply unit 120 in a conventional manner.

The system 100 includes a handle 130 for the device 8. The handle 130 is electrically connectable to the power supply unit 120 via an electrical connection 132, which in this embodiment is a cable. Both the handle 130 and the electrical connection 132 are preferably sterile, although they will become non-sterile during use. The handle is preferably light so as to pull as little in the device as possible, but should have some weight to stay steady on the table or in the user's hand. The power supply unit 120 can contain a safety circuit to protect from overvoltage. An adapter 125 may also be provided for connecting the handle 130 to the power supply unit 120. The power supply unit 120 can also include one or more sensors to measure impedance and output power. The power supply unit 120 can include a display or speaker for visual and/or auditory feedback of the procedure process. The power supply unit can be configured to shut off automatically at the procedure endpoint. The power supply unit can be configured so that there is no need for interaction with the settings during the procedure.

The RF hub is a non-sterile connection point for the handle and reference pad. It may be multi-use or disposable.

The power supply unit 120 contains electronics to monitor and modulate the electrical output to the reference pad 122 and device 8.

In FIG. 15, the device 8 is shown separately from the handle 130, although in use the device 8 will be partially inside the handle 130, as explained below.

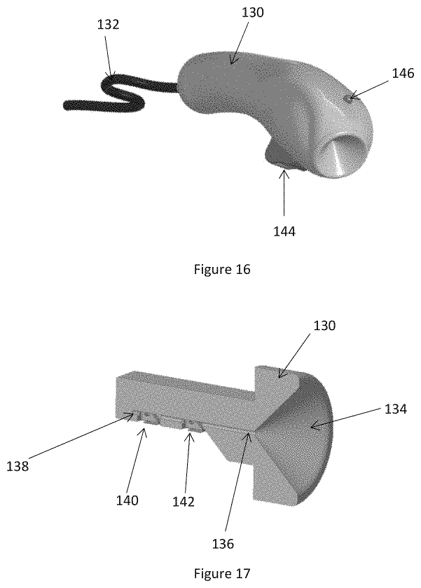

FIG. 16 shows a front perspective view of the handle 130.

FIG. 17 shows a cross-section view through the handle 130.

The handle 130 includes a funnel 134 at the distal end. At the base or apex of the funnel 134 is an opening of a passageway 136 which leads inside the handle 130.

The passageway 136 is configured to receive part of the guidewire proximal portion 12 so as to be ingress-proof as per 60601-2-2. The passageway 136 includes a proximal end 138 configured to receive the proximal end of the device 8, providing a pin vise. The pin vise can advantageously limit the backend connection of the core to the handle not to be thicker than the core outer diameter.

Along the passageway are provided a proximal connector 140 and a distal connector 142. In this embodiment, the connectors 140 and 142 are spring connectors, although other forms of connectors may be used. The connectors 140, 142 are configured to make electrical connection with the device 8 when the device is positioned in the passageway 136. In this embodiment, the connectors 140, 142 make electrical contact with the electrically conductive core 17.

The connectors 140, 142 are electrically connectable to the power supply unit 120 via the electrical connection 132.

An advantage of having two connectors is that this can allow measurement of connection resistivity for example by way of a resistivity sensor coupled between the connectors for example located in the power supply unit 120. They can also serve as a confirmation that the device is pushed correctly into the handle 130. They also provide a sturdy attachment. Nevertheless, in other embodiments, it is possible to have only one, or to have more than two, connectors.

As can be seen in FIG. 16, the handle 130 includes an actuator 144, which in this embodiment is in the form of an activation button or trigger. The actuator 144 is configured, in response to actuation thereof, to electrically connect the connectors 140, 142, to the electrical connection 132 in order to activate the device. In this embodiment, the actuator 144 can provide a momentary pushbutton type activation. The actuator 144 can be configured to disconnect the connectors 140, 142 from the electrical connection 132 in response to non-actuation of the actuator. In this way, the actuator provides an intuitive means of activating the device.

The handle 130 includes a notification element 146 configured to provide a notification in response to actuation of the actuator 144 so as to make clear that the device is active. In this embodiment, the notification element is a light emitter in the form of an LED which emits light while the actuator is actuated, but other notification elements can be provided, which can provide for example visual or audible notifications.

The handle 130 includes a housing which may be formed in an ergonomic shape, for example by moulding, so that the user may have a good grip, and preferably so that the user may manipulate the device with one hand. For intuitive use, in this embodiment the only elements for user interaction on the housing are the actuator 144 and notification element 146, and the actuator 144 is preferably ergonomically placed.

Manufacturing the device can including grinding the mandril which will form the core 17, welding and soldering the coils 19, 24, and applying the polymer jacket or insulation in manners known within guidewire manufacturing.

The proximal coil 27 can be loosened (stretched) ca 10% before being cut to length to provide some room for compressing or stretching to fit the mandril.

In some embodiments, the distal coil 19 is screwed on top of the proximal coil 27, the proximal coil 27 being stretched in its distal end to the same pitch as the distal coil 19.

The distal core portion 26 can be made the desired length by grinding off excess mandril after soldering the distal coil 19.

The system can be used in conjunction with conventional devices for intervention, for example microcatheters, introducers, wires, needles etc. in a suitably equipped interventional radiology or at a hospital.

To use the system, the user inserts the proximal end 12 of the device 8 into the passageway 136 of the handle 130 via the funnel 134. The funnel 134 serves to guide the proximal end 12 of the device 8 into the passageway 136.

The user pushes the device 8 fully back into the handle 130 so that the proximal end of the device 8 is held by the pin vise at the proximal end 138 of the passageway 136. This ensures that the device 8 has a sturdy attachment to the handle 130 and that the connectors 140, 142 make good electrical connections to the device 8.

The electrical connection 132 and the reference pad 122 are connected to the power supply unit 120.

The distal end of the device 8 is then inserted percutaneously into a blood vessel of a patient, typically an arterial blood vessel. The narrow and flexible distal end and energy delivery tip 16 of the device allows the device to pass through tortuous vessels, and the relative stiffness of the core 17, particularly the distal core portion 26, allows the distal end of the device 8 still to be effectively maneuvered through narrow and tortuous vessels despite the narrow and flexible character of the device. The correct positioning of the energy delivery tip 16 can be determined by making use of the radiopaque properties of the device 8, in particular of the electrically conductive element 19.

Meanwhile, the reference pad 122 is placed on the skin of the patient, preferably near to the blood vessel which it is desired to occlude.

Once the energy delivery tip 16 is positioned at the location of the vessel at which occlusion is desired, the shape of the energy delivery tip 16 ensures that the guidewire 10 engages the vessel wall at two contact points, at the proximal contact point 26 and the distal contact point 28.

Once the energy delivery tip 16 is positioned at the location of the vessel at which occlusion is desired, the power supply unit 120 is switched on. However, at this point the device is not active since the actuator 144 is not being actuated.

When it is desired to activate the device, the user actuates the actuator 144. This causes the connectors 140, 142 to be connected to the power supply unit 120, allowing the power supply unit 120 to supply electrical energy at RF frequencies through the system. In particular, an electrical circuit is formed from the power supply unit 120, through the electrical connection 132, through the core 17 (and optionally also through the proximal coil 27), through the distal core portion 26 and distal coil 19, through the patient's blood and tissue to the reference pad 122, and back to the power supply unit 120. This type of RF ablation apparatus is known as a monopolar system, where the anode terminal and the cathode pad are separate. In the embodiment of FIG. 15, the device 8 includes the anode terminal. More specifically, the energy delivery tip including the distal coil 19 and distal core portion 26 provide the anode terminal. The reference pad 122 includes the cathode pad. Electrical energy passing through the distal core portion 26 and the distal coil 19 passes (by conduction) through the patient to the cathode pad in the reference pad 122.

Actuation of the actuator 144 also causes the notification element 146 to provide notification to the user that the device is active, in this embodiment by emitting light.

In this embodiment, electrical energy passes simultaneously through the distal core portion 26 and the electrically conductive element 19 in parallel, the electrically conductive element 19 thereby compensating for the lesser conductive properties of the narrow and stiff distal core portion 26, and increasing the current capacity and total power which can be provided by the device 8.

While the one or more sensors in the power supply unit 120 determine that the impedance is within an operable range, the power supply unit is configured to moderate the output power and gradually to ramp it up.

As electrical energy is passed through the guidewire 10, the energy results in localised heating of the proximal and distal tip ends as discussed above. The contact of the energy delivery tip with the vessel wall at at least two locations causes ablation of the vessel wall 15a, creating a thrombus and fibrotic response resulting in embolization of the target vessel, which is particularly effective where the contact points are at or near the proximal and distal tip ends owing to the increased heating there. The system is configured only to affect the vessel wall and not to affect surrounding tissue at a clinically significant level. Appropriate parameters for operation for this can be determined by the skilled person in view of the procedure desired.

Preferably, the system is configured to cause occlusion at the target vessel within 3 minutes of energy delivery and the occlusion should be persistent at 7 days.

In response to the one or more sensors detecting an impedance shift, the power supply unit 120 can be configured to modulate the output power to an impedance dependent lower level for a predetermined amount of time. The appropriate parameter for this can be determined by the skilled person.

The power supply unit can then be configured to indicate to the user that the end of the procedure has been reached. There may then be an optional repositioning and/or channel treatment.

The precise details of the method of treatment may be varied by the skilled person in accordance with the desired treatment to be delivered. In addition, it is not necessary for the power supply unit 120 to include an impedance sensor or an output power sensor; the power supply unit 120 may operate on the basis of different parameters or may include no sensors if the procedure is to be left to the user's judgment.

Once the device has created the occlusion, it can be retracted and removed completely from the occlusion site of the vessel, leaving no implant behind and without tearing open or rupturing the newly formed occlusion. The user can then reposition the device 8 at another target vessel or another location of the vessel at which further occlusion is desired and repeat the procedure for causing ablation and/or embolisation. This enables the device to target multiple vessels in the same session using the same device.

The device shape and geometry ensures that the device may function as a guidewire allow it to be inserted into and cause embolization of very small vessels. In particular, the device does not require a microcatheter to track to the treatment target.

Although in the above described embodiments, the majority of the electronics are in the power supply unit, the electronics can be in either or both of the handle and power supply unit.

In the above described embodiments, the guidewire distal portion 14 and guidewire tip 16 are shaped such that that the device contact or engages the vessel wall at two contact points. However, it is envisaged that the guidewire distal portion 14 and/or the energy delivery tip 16 may be shaped such that the device 8 contacts the vessel wall in more than two points.

In the system 100 described above, RF ablation using a monopolar system is described. However, in other embodiments, RF ablation using a bipolar system may be envisaged. In such embodiments, the energy delivery tip core 26 includes both the anode and the cathode terminals spaced longitudinally from one another on the guidewire 10. The cathode may for example be located on top of the insulation 22. The anode and cathode may be interchanged in other embodiments.

In the above described embodiments, the guidewire proximal portion 12 and the guidewire distal portion 14 are straight, while the energy delivery tip 16 is curved. However, in other embodiments, the guidewire distal portion 14 may be curved or have a curved unbiased configuration; while the energy delivery tip 16 is straight or have a straight unbiased configuration. In embodiments where the guidewire distal portion 14 is curved, the curve of the guidewire distal portion 14 defines a tangent or a chord which determines the first direction. The energy delivery tip 16 extends in a second direction which is different from the first direction, such that the distal tip end 20 is offset from the proximal tip end 18. The curved shape of the guidewire distal portion 14 can ensure that the energy delivery tip contacts the vessel wall 15a at two points, advantageously with one contact point being at the guidewire distal portion 14 and the other contact point being at the distal tip end 20. In such embodiments, the guidewire distal portion 14 is preferably curved with a constant radius of curvature and the guidewire distal portion 14 curves in a single plane. In other words, the tangent lines defined by the curvature of the guidewire distal portion 14 lie in a single plane. The guidewire distal portion 14 is also preferably curved such that it does not curve back or bend back on itself.

In other embodiments, both the guidewire distal portion 14 and the energy delivery tip 16 may be straight or have straight unbiased configurations. In such embodiments; the guidewire distal portion 14 extends in a first direction and the energy delivery tip 16 extends at an angle from the guidewire distal portion 14 such that the energy delivery tip 16 extends in a second direction which is different from the first direction of the guidewire distal portion 14 and such that the distal tip end 20 is offset from the proximal tip end 18. This can mean that the energy delivery tip can contact the vessel wall at two points, advantageously with one contact point being at the proximal tip end and the other contact point being at the distal tip end.

In other embodiments, both the guidewire distal portion and the energy delivery tip can be curved or have a curved unbiased configuration in the manners discussed above.

All optional and preferred features and modifications of the described embodiments and dependent claims are usable in all aspects of the invention taught herein. Furthermore, the individual features of the dependent claims, as well as all optional and preferred features of the dependent claims, as well as all optional and preferred features and modifications of the described embodiments are combinable and interchangeable with one another. Features of the device may be combined with or modified by any of the features disclosed in UK application number [ ] filed on even date and entitled Energy Delivery Device for Endovascular Occlusion, the contents of all of which are hereby incorporated by reference.

The disclosures in the abstract accompanying this application are incorporated herein by reference.

1. An endoluminal energy delivery device including a proximal end and a distal end; an electrically conductive core extending from the proximal to the distal end: an energy delivery tip at the distal end; the energy delivery tip including a distal core portion and an electrically conductive element disposed coaxially around the distal core portion and electrically coupled thereto in parallel; wherein the distal core portion is made of a first material and the electrically conductive element is made of a second material, wherein the second material has a higher conductivity than the first material; whereby electrical energy supplied to the delivery device passes simultaneously through the distal core portion and the electrically conductive element.

2. An endoluminal energy delivery device according to claim 1, wherein the electrically conductive element is a helical wire coil.

3. An endoluminal energy delivery device according to claim 1, wherein the distal core portion is made of a first material and the electrically conductive element is made of a second material, wherein the second material has a greater flexibility than the first material.

4. An endoluminal energy delivery device according to claim 1, wherein the distal core portion has a greater stiffness than a stiffness of the electrically conductive element.

5. An endoluminal energy delivery device according to claim 1, wherein the distal core portion is made of nickel titanium alloy or stainless steel.

6. An endoluminal energy delivery device according to claim 1, wherein the distal core portion is substantially free of copper, silver and gold.

7. An endoluminal energy delivery device according to claim 1, wherein the electrically conductive element is made of platinum, gold, silver or alloys thereof.

8. An endoluminal energy delivery device according to claim 1, wherein the electrically conductive element is made of or includes a radiopaque constituent.

9. An endoluminal energy delivery device according to claim 1, wherein the electrically conductive element is made of a material including tungsten.

10. An endoluminal energy delivery device according to claim 1, including an electrically insulating sheath disposed coaxially around the core from the proximal end of the device to a proximal end of the distal core portion.

11. An endoluminal energy delivery device according to claim 1, wherein the core includes a tapered portion tapering towards a distal extremity of the device.

12. An endoluminal energy delivery device according to claim 11, wherein the tapered portion is proximal of the distal core portion.

13. An endoluminal energy delivery device according to claim 11, including a second electrically conductive element, the second electrically conductive element being disposed coaxially around the tapered portion.

14. An endoluminal energy delivery device according to claim 13, wherein the second electrically conductive element is a wire coil.

15. An endoluminal energy delivery device according to claim 13, wherein the second electrically conductive element is a tubular coil made of stainless steel.

16. An endoluminal energy delivery device according to claim 13, wherein the second electrically conductive element is electrically coupled to the core in parallel; whereby electrical energy supplied to the delivery device passes simultaneously through the tapered portion and the second electrically conductive element.

17. An endoluminal energy delivery device according to claim 13, including an electrically insulating sleeve disposed coaxially over the second electrically conductive element.

18. An endoluminal energy delivery device according to claim 1, including a rounded tip at the distal end of the device, the distal core portion and electrically conductive element being coupled to the rounded tip.

19. An endoluminal energy delivery device according to claim 1, wherein the rounded tip is electrically conductive, the distal core portion and electrically conductive element being connected together electrically through the rounded tip.

20. An endoluminal energy delivery device according to claim 1, wherein the electrically conductive element is coated with an heat conductive material.

21. An endoluminal energy delivery device according to claim 1, wherein the energy delivery tip is non-stick with respect to charred blood.

22. An endoluminal energy delivery device according to claim 1, wherein the electrically conductive element is coated with a layer of parylene.

23. An endoluminal energy delivery device according to claim 1, wherein the electrically conductive element is coated with a layer of chromium nitride (CrN).

24. An endoluminal energy delivery device according to claim 1, wherein the device is a radiofrequency ablation electrode.

25. An endoluminal energy delivery device according to claim 1, wherein the device is a radiofrequency ablation electrode of a monopolar vessel ablation system.
